(19) United States
(12) Patent Application Publication
(10) Pub. No.: US 2016/0220684 A1
(43) Pub. Date: Aug. 4, 2016

(54) COMPOSITION AND METHOD FOR RELIEVING PAIN AND INFLAMMATION, TREATMENT OF ERECTILE DYSFUNCTION, AND TREATMENT OF ACNE

(71) Applicants: Robert E. Nordquist, Oklahoma City, OK (US); James A. Rosacker, Oklahoma City, OK (US); Patrick J. Downes, Oklahoma City, OK (US)

(72) Inventors: Robert E. Nordquist, Oklahoma City, OK (US); James A. Rosacker, Oklahoma City, OK (US); Patrick J. Downes, Oklahoma City, OK (US)

(21) Appl. No.: 14/933,531

(22) Filed: Apr. 19, 2016

Related U.S. Application Data
(60) Provisional application No. 62/075,929, filed on Nov. 6, 2014.

(51) Int. Cl. A61K 47/36; A61K 31/451; A61K 9/00; A61K 47/10; A61K 31/10; A61K 31/16; A61K 45/06; A61K 31/045; A61K 31/513; A61K 31/407; A61K 31/192

(52) U.S. Cl. CPC A61K 47/36; A61K 31/407; A61K 31/451; A61K 9/0014; A61K 47/10; A61K 31/192; A61K 31/16; A61K 45/06; A61K 31/045; A61K 31/513; A61K 31/10

(57) ABSTRACT

Transdermal compositions including chitosan, glycated chitosan, or a combination thereof are provided for use in treatment methods wherein the compositions are externally applied for relieving pain and inflammation, treating erectile dysfunction, treating acne, causing hair growth, and for veterinary treatment of animals for relieving pain and inflammation.

### RELATED CASE

This application claims the benefit of U.S. Provisional Patent Application Ser. No. 62/075,929 filed on Nov. 6, 2014, and incorporates said provisional application by reference into this disclosure as if fully set out at this point.

### FIELD OF THE INVENTION

The present invention relates to transdermal compositions and methods for reducing or eliminating pain and inflammation. The present invention also relates to compositions and methods for treating erectile dysfunction, hair growth and veterinary applications. In addition, the present invention relates to compositions and methods for treating acne.

### BACKGROUND OF THE INVENTION

The immune inflammatory response is the most common cause of pain in human soft tissues. Most inflammatory conditions are musculoskeletal or orthopedic in nature and are usually localized, such as, for example, in cases of injury, arthritis, affliction and/or infection.

Heretofore, most pharmacy prepared transdermal compositions have only been nominally effective for treating such conditions. In addition, these compositions (a) commonly contain many redundancies in active ingredients, (b) do not contain sufficient concentrations of active components, (c) involve complex combinations, (d) often include clinically irrelevant ingredients, and/or (d) are typically quite costly, particularly for elderly patients who are commonly afflicted with inflammatory ailments.

Consequently, a need currently exists for a more effective transdermal composition and method for relieving or eliminating body pain and inflammation. The composition also preferably (a) will be easy and convenient to apply to the painful area, (b) will have a soft, pleasant feel to the skin, (c) will not have an unpleasant smell, and (d) will be less complex and less costly and very safe.

A need also exists for a composition and method which are effective for treating erectile dysfunction (ED) but do not require the use of ED medications in amounts that will interfere with the heart medications used by some patients.

In addition, a continuing need exists for acne treatment compositions and methods which are highly effective and are antimicrobial, invisible, odorless and convenient to use.

Chitosan (referred to herein as “non-glycated chitosan”) is a linear polysaccharide composed of randomly distributed β-(1-4)-linked D-glucosamine and N-acetyl-D-glucosamine. It is a product of chitin, derived principally from shrimp shells and tails. In medicine, chitosan can be useful in bandages to reduce bleeding and as an antibacterial agent. Chitosan can also be used to help deliver drugs through the skin.

Glycated chitosan is an immuno-adjuvant for vaccines used in cancer immunotherapy. Glycated chitosan is a water soluble compound which is formed by the attachment of galactose molecules to chitosan molecules. Glycated chitosan stimulates the patient's immune system and increases the patient's response to the cancer vaccine. Glycated chitosan is typically given to the patient through intratumoral administration following laser irradiation of the tumor lesions.

Neither non-glycated chitosan nor glycated chitosan has been used heretofore for alleviating body pain and inflammation or for treatment of erectile dysfunction or acne or veterinary applications.

Ketorolac tromethamine is a nonsteroidal anti-inflammatory drug (NSAID), used as an analgesic for short-term treatment of moderate to severe pain, usually before or after surgical or other medical procedures. Ketorolac tromethamine inhibits the bodily synthesis of prostaglandins. Heretofore, it has been advised that ketorolac tromethamine not be used for mild or long-term painful conditions such as arthritis.

The chemical name for ketorolac tromethamine is (±)-5-benzoyl-2,3-dihydro-1H-pyrrolizine-1-carboxylic acid, compounded with 2-amino-2-(hydroxymethyl)-1,3-propanediol. Ketorolac tromethamine is commercially available under the trade names TORADOL, ACULAR, and SPRIX. Ketorolac tromethamine has been administered by injection into a muscle or vein and has also been available in forms approved for oral, ocular, and nasal administration.

(RS)-1-butyl-N-(2,6-dimethylphenyl) piperidine-2-carboxamide is a local anesthetic drug which is commercially available under the trade names BUPIVACAINE, MARCAIN, MARCAINE, SENSORCAINE, and VIVACAINE.

2-(diethylamino)-N-(2,6-dimethylphenyl) acetamide is a local anesthetic available under the names LIDOCAINE, XYLOCAINE, and LIGNOCAINE.

(6R-trans)-6-(1,3-benzodioxol-5-yl)-2,3,6,7,12,12a-hexahydro-2-methyl-pyrazino [1′, 2′:1,6] pyrido[3,4-b]indole-1,4-dione is a medication for treating ED which is commercially available under the name TADALAFIL. TADALAFIL and other medications such as SILDENAFIL CITRATE, VARDENAFIL, etc. affect the nitric oxide cycle that regulates penile blood flow and erection.

### SUMMARY OF THE INVENTION

The present invention satisfies the needs and alleviates the problems discussed above.

In one aspect, there is provided a transdermal composition for relieving pain and inflammation comprising: non-glycated and/or glycated chitosan; ketorolac tromethamine; and (RS)-1-butyl-N-(2,6-dimethylphenyl) piperidine-2-carboxamide. The composition preferably also comprises glycerin and water.

In another aspect, there is provided an alternative transdermal composition for relieving pain and inflammation comprising Tris(2-hydroxyethyl)ammonium 2-hydroxybenzoate (i.e., Trolamine salicylate) and 2-(diethylamino)-N-(2,6-dimethylphenyl) acetamide (e.g., LIDOCAINE). It is additionally preferred that this alternative composition comprise an aqueous solution of chitosan (non-glycated, glycated, or a combination thereof) and/or glycerin.

In another aspect, there is provided a method of relieving pain and inflammation comprising the step of externally (topically) applying the inventive transdermal composition to the skin in the affected area. Preferably, the inventive transdermal composition is externally applied to the skin manually or by spraying or misting.

The inventive composition and method are surprisingly and unexpectedly effective for reducing or eliminating pain and inflammation. As will be discussed below, tests have confirmed not only that the inventive composition and method are highly effective for treating more common joint and muscle pains and inflammation, but that the inventive composition and method are highly effective even for extreme ailments and conductions such as burns, shingles, diabetic neuropathy, and rheumatoid arthritis, non-traumatic musculoskeletal conditions, sprains, strains, muscle spasms, sciatica, myofascitis, nerve contusions, post-operative incisions, carpal tunnel syndrome, peripheral neuropathies, sunburns, cellulitis, abscesses, insect bites, herpetic lesions, fever blisters, pressure sores, poison ivy, etc. Moreover, the inventive composition is composed entirely of FDA approved components and produces no undesirable systemic or other side effects, and eliminates the liver “first pass through” effect.

In addition to the use for human patients, these inventive transdermal compositions can also be used in veterinary treatments for animals for relief of pain and inflammation. An alternative composition for veterinary use preferably comprises: (a) chitosan, glycated chitosan, and/or derivations thereof and (b) dimethyl sulfoxide (DMSO). The composition can also include ketorolac tromethamine, 2-(diethylamino)-N-(2, 6-dimethylphenyl) acetamide (e.g., LIDOCAINE), glycerin, and/or water.

The inventive transdermal composition for relieving pain and inflammation initially dulls the pain within a few minutes and helps reduce inflammation and swelling. The composition works to relieve both the immediate pain and the underlying source of the pain. The composition also promotes healing and tissue reorganization and is naturally antimicrobial.

In addition, because the composition is not taken internally, it does not alter the patient's ability to continue normal work, unlike some systemic medications. Further, the inventive transdermal composition requires a much smaller amount of the active components therein for treating symptoms of pain and inflammation topically, as compared to systemic medications, particularly narcotics. The inventive composition takes the active ingredients directly to the source of discomfort, rather than to the rest of the body. This also reduces the likelihood for complications and side effects.

In another aspect, there is provided a transdermal ED composition for treating erectile dysfunction comprising: non-glycated and/or glycated chitosan; 2-(diethylamino)-N-(2,6-dimethylphenyl) acetamide; and TADALAFIL, SILDENAFIL CITRATE, VARDENAFIL, or other compound or composition affecting the nitric oxide cycle that regulates penile erection.

In another aspect, there is provided a method of treating erectile dysfunction comprising the step of externally (topically) applying the inventive transdermal composition to the penis. Preferably, the inventive transdermal composition is externally applied to the penis manually or by spraying, misting, or by other liquid application techniques.

In another aspect, there is provided a transdermal composition for hair growth comprising (a) chitosan, glycated chitosan or derivations thereof and Minoxidal. The composition can also include glycerin and/or water.

In another aspect there is provided a method of treating a patient for hair growth comprising externally (topically) applying the inventive transdermal hair growth composition to the patient's scalp, e.g. manually or by misting or spraying.

In another aspect, there is provided a composition for treating acne comprising: an aqueous solution of chitosan (non-glycated, glycated, or a combination thereof); ethanol; and ketorolac tromethamine.

In another aspect, there is provided a method of treating acne comprising spaying, misting, manually applying, or otherwise applying the inventive acne treatment composition to the face or other affected area of the skin.

Further aspects, features, and advantages of the present invention will be apparent to those of ordinary skill in the art upon reading the following detailed description of the preferred embodiments.

### DETAILED DESCRIPTION OF THE PREFERRED EMBODIMENTS

The inventive transdermal composition for relieving pain and inflammation preferably comprises: from about 20 to about 60 parts by volume of an aqueous solution of chitosan (non-glycated, glycated, or a combination thereof); at least 2 parts by volume (more preferably from about 5 to about 30 parts by volume) ketorolac tromethamine; at least 2 parts by volume (more preferably from about 5 to about 30 parts by volume) (RS)-1-butyl-N-(2,6-dimethylphenyl) piperidine-2-carboxamide; and from about 5 to about 30 parts by volume glycerin.

The inventive transdermal composition for relieving pain and inflammation most preferably comprises: about 50 parts by volume of an aqueous solution of chitosan (non-glycated, glycated, or a combination thereof); about 20 parts by volume ketorolac tromethamine; about 20 parts by volume (RS)-1-butyl-N-(2,6-dimethylphenyl) piperidine-2-carboxamide; and about 10 parts by volume glycerin.

The aqueous chitosan solution preferably comprises ≦1% by volume non-glycated chitosan, glycated chitosan, or a combination thereof. More preferably, the aqueous chitosan solution comprises from about 0.01 to about 1.0% by volume chitosan (non-glycated, glycated, or a combination thereof) and from about 99.99 to about 99% by volume water. Most preferably, the aqueous chitosan solution comprises about 0.25% by volume chitosan (non-glycated, glycated, or a combination thereof) and about 99.75% by volume water. The water used in the aqueous glycated chitosan solution is preferably deionized, sterile water.

The chitosan provides anti-microbial and preservative properties to the composition and assists in driving the transdermal flux of the composition components into and through the skin to the inflamed tissues.

The ketorolac tromethamine component used in the inventive transdermal composition for relieving pain and inflammation is a non-steroidal anti-inflammatory which inhibits the release of prostaglandins and thereby provides significant relief of muscle and bone pain. In its parenteral form as used in the inventive transdermal composition, ketorolac tromethamine is more efficacious for controlling inflammatory pain than most if not all other anti-inflammatory products available and is generally superior to and safer than intra-venous narcotics for post-surgical pain. In an article published May 28, 2015 in the New England Journal of Medicine relating to intractable pain control in patients with metastatic cancer who were refractory to opiods, keterolac was the only effective non-opiod analgesic. Given its nature as a COX-2 inhibiter, the ketorolac tromethamine component of the inventive transdermal composition will sequentially suppress the inflammatory condition by deactivating the MI cytotoxic macrophages which produce and secrete the noxious proteins which create the inflammatory pain. Ketorolac tromethamine is preferably present in the inventive transdermal composition for relieving pain and inflammation in an amount in the range of from about 0.5 to about 15 mg per ml of the total volume of the composition and is most preferably present in an amount of about 3 mg/ml.

(RS)-1-butyl-N-(2,6-dimethylphenyl) piperidine-2-carboxamide (referred to hereinafter by the trade name BUPIVACAINE) operates in the inventive composition for relieving local pain. BUPIVACAINE quickly penetrates the skin to provide local relief of inflammatory pain. The BUPIVACAINE component will not only rapidly reduce or often eliminate the pain at the nidus, but will as well reduce or eliminate the referred pain for a period of hours. (RS)-1-butyl-N-(2,6-dimethylphenyl) piperidine-2-carboxamide is preferably present in the inventive transdermal composition for relieving pain and inflammation in an amount in the range of from about 0.5 to about 15 mg per ml of the total volume of the composition and is most preferably present in an amount of about 3 mg/ml.

The combination of ketorolac tromethamine and BUPIVACAINE used in the inventive composition for relieving pain and inflammation provides rapid pain relief while also progressively suppressing and reducing or eliminating the inflammatory source of the pain, tenderness, and swelling with each succeeding application.

The glycerin component of the inventive transdermal composition enhances the softness and feel of the inventive composition and softens the patient's skin. In addition, the glycerin component operates as a thickening or holding agent for the inventive composition which assists in holding the composition in place when applied so as to minimize run off of the composition on the patient's skin.

When formulated as described above, the components of the inventive transdermal composition are miscible and will remain in solution. Consequently, in accordance with the inventive method, the inventive composition can be conveniently applied to the skin in the area of pain and inflammation by spaying or misting, or by any other desired liquid application technique. The inventive composition can then be reapplied as needed up to as often as once every 1-3 hours.

### EXAMPLE

Over a period of 20 months, approximately 250 patients suffering from the various conditions listed below were each provided a ⅓ oz. spay mist bottle of the inventive transdermal composition for relieving pain and inflammation. These were issued on the occasion of each office encounter over the 20 months of testing. The inventive composition comprised: (a) 50% by volume of a 0.25% to 0.5% aqueous solution of non-glycated chitosan; (b) 20% by volume ketorolac tromethamine (i.e., 3 mg per ml of the final mixture); (c) 20% by volume BUPIVACAINE (i.e., 3 mg per ml of the final mixture); and (d) 10% by volume glycerine.

The patients were instructed to apply the inventive composition to the affected area not more often than once every 1 to 3 hours, using only a single squirt of the spray mist (approximately 1/9 ml) per each application. In each case, the inventive treatment resulted in the significant reduction or elimination of pain and inflammation in the treated area.

Patient Diagnosis:
Arthropathy, NEC & Arthropathy shoulder
CTS/Arthropathy NEC
Pain in joint, lower leg
Synovitis, NOS & Effusion
Arthropathy NEC
Osteoarthritis, Pelvis & Synovitis
Arthropathy NEC, Neuritis, Dermatitis
Myalgia, Arthropathy NEC, Lesion Med Nerve
Pain in joint, lower leg
Enthesopathy, lower leg
Fasciitis, NOS
Synovitis NEC, Arthropathy NEC, Pain lower leg
Contusion elbow & knee, Arthropathy NEC
Arthropathy, NEC
Osteoarthritis, knee & Arthropathy, NEC
Pain in joint, lower leg & Synovitis, NEC
Achilles tendinitis & Synovitis, NEC
Lumbar Disc Syndrome & Arthropathy, NEC
Herpes zoster NOS
Osteoarthritis, hand & CTS
Medial Epicondylitis & Arthropathy, NEC
Lesion Plantar Nerve
Arthropathy, NEC
Pain in joint, hand & Disturbance of skin sensation
Arthropathy, NEC
Fasciitis, NOS
Synovitis, NOS
Arthropathy, NEC
Fx closed metatarsal
Sprain/Strain neck and shoulder
Tenosynovitis
Mononeuritis
Arthropathy, NEC Fx closed radius
Osteoarthrosis, lower leg & Effusion
Synovitis NEC, & Effusion
Pain in joint lower leg
Pain in joint lower leg
Lumbar Sprain/Strain
Arthropathy, NEC & Neuralgia
Pain in joint lower leg & Synovitis
Fasciitis & Lumbago
Osteoarthrosis, hand/FX Ulna & Distal Radius
Capsulitis/Osteoarthrosis, shoulder
Arthropathy, NEC
Fasciitis
Capsulitis, shoulder
CTS/ Other lesion of median nerve
Arthropathy, NEC/Radiculitis
Arthropathy, NEC/Tenosynovitis, hand
Arthropathy, NEC
Arthropathy, NEC
Lumbar Disc Syndrome & Arhtropathy, NEC, Radiculopathy
Impingement, shoulder/Arthropathy, NOS
Arthropathy, NEC/Radiculitis/Lumbar S/S
Lateral Epicondylitis
Arthropathy, NEC
Lateral Epicondylitis/Lesion of radial nerve
Tenosynovitis, ankle/Arthropathy, ankle
Pain in joint, hand/Second degree burn, arm
Arthropathy, NEC
Tenosynovitis, hand/wrist
Arthropathy, NEC/Capsulitis/Achilles tendinitis
Arthropathy, NEC
Arthropathy, NEC
Cervical S/S & Shoulder S/S
Synovitis, NOS
Myofasciitis/ Arthropathy, NEC
Tenosynovitis, foot/ankle & Arthropathy, ankle
Pain in joint, ankle
Pain in joint, knee/Synovitis
Osgood-Slaughter apophysitis/pain in joint, knee
Contusion of knee
Arthropathy, NEC
Achilles tendinitis
Osteoarthrosis, NOS/Arthropathy, NEC
Enthesopathy, hip
Fx closed ulna, Olecranon process
Fx closed Humerus, Surgical neck
Contusion of wrist/Sprain/Strain of wrist
Pain in joint, hand
Pain in joint, shoulder/Arthropathy, NEC
CTS/Lateral Epicondylitis/Capsulitis
Ankle Sprain/Strain NOS
Osteoarthrosis, shoulder/Capsulitis
Pain in joint, lower leg.
Contusion of knee
Arthropathy, NEC/Tenosynovitis, hand/wrist NOS
Osteoarthrosis, NOS upper arm
Temporal-mandibular joint (TMJ) synovitis/arthritis

Results to Date:

Approximately 250 patents

Acute and persistent (chronic or recurrent) conditions

The contents of the over 2500 5 cc sample bottles issued were completely used

Therefore, the approximate number of applications in the study to date is 75,000 over 20 months

Each subject was interviewed directly and in person by the treating physician director of the investigation

Based on these interviews:
The EFFICACY of the topical was overwhelmingly expressed as excellent for control or elimination of the presenting inflammatory symptoms
There were NO reports of adverse side effects, either locally or systemically

In an alternative embodiment of the inventive transdermal composition for relieving pain and inflammation, the composition preferably comprises: from about 0.1% to about 10% (more preferably about 3%) by volume Tris(2-hydroxyethyl)ammonium 2-hydroxybenzoate (i.e., Trolamine salicylate) and from about 0.1% to about 4% (more preferably about 1%) by volume 2-(diethylamino)-N-(2,6-dimethylphenyl) acetamide (e.g., LIDOCAINE). It is additionally preferred that this alternative composition for relieving pain and inflammation include (a) from about 20% to about 60% (more preferably about 50%) by volume of an aqueous solution of chitosan (non-glycated, glycated, or a combination thereof) as described above and/or (b) from about 5% to about 30% (more preferably about 10%) by volume glycerin. In either case, the remaining volume of the composition to provide a total volume of all components of 100% will preferably be water.

The inventive transdermal composition for treating erectile dysfunction (ED) preferably comprises: (a) from about 20 to about 60 parts by volume of an aqueous solution of chitosan (non-glycated, glycated, or a combination thereof) as described above and (b) an amount of TADALAFIL, SILDENAFIL CITRATE, VARDENAFIL, and/or other compound or composition affecting the nitric oxide cycle that regulates penile erection. The composition can also include at least 0.1 parts by volume (more preferably from about 0.1 to about 4.0 parts by volume and most preferably 1 part by volume) 2-(diethylamino)-N-(2,6-dimethylphenyl) acetamide (e.g., LIDOCAINE).

The amount of TADALAFIL, SILDENAFIL CITRATE, VARDENAFIL, and/or other compound or composition affecting the nitric oxide cycle that regulates penile erection, used in the inventive ED composition can be any amount which is effective for safely treating erectile dysfunction and which will remain in solution in the inventive composition. Moreover, in the inventive ED composition, the amount of the component or combination of components affecting the nitric oxide cycle that regulates penile erection can surprisingly be an amount which is less than the amounts required for oral administration which interfere with heart medications.

The amount of TADALAFIL, SILDENAFIL CITRATE, VARDENAFIL, and/or other compound or composition affecting the nitric oxide cycle that regulates penile erection, used in the inventive ED composition will preferably be an amount in the range of from about 0.1 to about 10.0 micrograms or to about 5.0 mg (more preferably about 1 mg) per ml of the total volume of the ED composition.

The component used in the inventive ED composition for affecting the nitric oxide cycle that regulates penile erection will preferably be TADALAFIL

When formulated as described above, the components of the inventive transdermal ED composition are miscible and will remain in solution. Consequently, in accordance with the inventive method, the inventive composition can be conveniently applied to the penis manually or by spraying , misting, or any other desired application technique.

The inventive transdermal composition for hair growth preferably comprises: (a) chitosan, glycated chitosan and/or derivations thereof and (b) Minoxidil. The composition is preferably an aqueous composition and can include glycerin. The composition can be applied to the scalp manually or by misting or spraying, or by any other desired technique.

The inventive transdermal veterinary composition for relieving pain and inflammation in animals preferably comprises: (a) chitosan, glycated chitosan, and/or derivation thereof and optionally (b) dimethylsulfide. The composition is preferably an aqueous composition and can also include glycerin, ketorolac tromethamine, and/or 2-(diethylamino)-N-(2,6-dimethylphenyl) acetamide (e.g. LIDOCAINE). The veterinary composition can be applied by spraying and rubbing to bypass the animal hair.

The inventive composition for treating acne preferably comprises: an amount of an aqueous solution of chitosan (non-glycated, glycated, or a combination thereof) as described above sufficient to provide a non-glycated and/or glycated chitosan concentration of from about 0.1% or from about 0.25% to about 0.5% (more preferably 0.1%) by volume; from about 1% to about 10% (more preferably about 2%) by volume ethanol (preferably absolute ethanol); and from about 0.1 to about 3 mg (more preferably about 1 mg) per ml of the total composition of ketorolac tromethamine. The remaining volume of the composition to provide a total volume of all components of 100% will preferably be water.

In accordance with the inventive method, the inventive composition for treating acne can be conveniently applied as a lotion or by spaying or misting, or by any other desired application technique, to the face or other affected area of the skin.

Thus, the present invention is well adapted to carry out the objects and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within this invention as defined by the claims.

What is claimed is:

1. A transdermal composition comprising:
non-glycated chitosan, glycated chitosan, or a combination thereof;
ketorolac tromethamine; and
(RS)-1-butyl-N-(2,6-dimethylphenyl) piperidine-2-carboxamide.

2. The transdermal composition of claim 1 further comprising glycerin.

3. A method of relieving pain or inflammation comprising externally applying the transdermal composition of claim 1 to a person's skin in an area of pain or inflammation.

4. A transdermal composition comprising:
Tris (2-hydroxyethyl) ammonium 2-hydroxybenzoate;
2-(diethylamino)-N-(2, 6-dimethylphenyl) acetamide; and
non-glycated chitosan, glycated chitosan, or a combination thereof.

5. The transdermal composition of claim 4 further comprising glycerin.

6. A method of relieving pain or inflammation comprising externally applying the transdermal composition of claim 4 to a person's skin in an area of pain or inflammation.

7. A transdermal composition for treating erectile dysfunction comprising:
non-glycated chitosan, glycated chitosan, or a combination thereof;
2-(diethylamino)-N-(2,6-dimethylphenyl) acetamide; and
a compound which affects a human nitric oxide cycle to produce a penile erection.

8. A method of treating erectile dysfunction comprising externally applying the transdermal composition of claim 7 to a person's penis.

9. A composition for treating acne comprising:
non-glycated chitosan, glycated chitosan, or a combination thereof;
ethanol; and
ketorolac tromethamine.

10. A method of treating acne comprising externally applying the composition of claim 9 to an acne affected area of a person's skin.

11. A composition for hair growth comprising:
chitosan, glycated chitosan or a combination thereof and
minoxidil.

12. A method of hair growth comprising externally applying the composition of claim 11 to a person's scalp.

13. A transdermal veterinary composition for relieving pain or inflammation in animals comprising:
chitosan, glycated chitosan or a combination thereof and
dimethyl sulfoxide.

14. A method of relieving pain or inflammation for animals comprising externally applying the transdermal veterinary composition of claim 13 to an animal's skin.
